(19) United States
(12) Patent Application Publication
(10) Pub. No.: US 2024/0108504 A1
(43) Pub. Date: Apr. 4, 2024

(54) NEEDLE FOR ATTACHMENT TO OPHTHALMIC SURGICAL HANDPIECES

(71) Applicant: Bausch + Lomb Ireland Limited, Dublin (IE)

(72) Inventors: Brian MCCARY, Clayton, MO (US); Toh Seng GOH, Wildwood, MO (US); Todd SMITH, Sharpsburg, GA (US)

(21) Appl. No.: 18/376,693

(22) Filed: Oct. 4, 2023

Related U.S. Application Data
(60) Provisional application No. 63/413,028, filed on Oct. 4, 2022.

(51) Int. Cl. A61F 9/007; A61B 90/00

(52) U.S. Cl. CPC A61F 9/00745; A61B 90/00; A61B 2217/002

(57) ABSTRACT

An ultrasonic needle for attachment to an ophthalmic surgical handpiece includes a cannula having a proximal end and a distal end. The proximal end of the cannula is configured for attachment to an ophthalmic surgical handpiece. The distal end of the cannula includes a tip with at least one port in communication with a lumen extending through the cannula. A flexible member (e.g., a flexible loop, a flexible button, etc.) is coupled to the distal end of the cannula for tissue manipulation.

### FIELD

The present disclosure generally relates to needles for attachment to ophthalmic surgical handpieces and related systems, and more particularly, to needles including structures for manipulating tissues of the eye.

### BACKGROUND

This section provides background information related to the present disclosure which is not necessarily prior art.

Ophthalmic surgery often involves cutting away or emulsifying tissues that need to be removed from the eye, such as in cataract surgery. One known technique is phacoemulsification. This technique involves using high frequency ultrasound energy transmitted through a handpiece into a phacoemulsification needle to emulsify the affected tissue. The emulsified tissue is removed from the eye through an aspiration path. During surgery, the phacoemulsification needle may need to work very close to highly delicate parts of the eye, such as the retina and posterior capsule, without damaging those parts of the eye.

### SUMMARY

This section provides a general summary of the disclosure, and is not a comprehensive disclosure of its full scope or all of its features.

Example embodiments of the present disclosure generally relate to needles for attachment to ophthalmic surgical handpieces. In one example embodiment, an ultrasonic needle generally includes a cannula having a proximal end and a distal end. The proximal end of the cannula is configured for attachment to an ophthalmic surgical handpiece. The distal end of the cannula includes a tip with at least one port in communication with a lumen extending through the cannula. A flexible member (e.g., a flexible loop, a flexible button, etc.) is coupled to the distal end of the cannula for tissue manipulation.

In another example embodiment, an ophthalmic surgical handpiece is provided. The handpiece generally includes a housing having a distal end. A cannula is attached to the distal end of the housing and includes a distal tip with at least one aspiration port. A vibration source is disposed within the housing for vibrating the distal tip of the cannula. A flexible member (e.g., a flexible loop, a flexible button, etc.) is coupled to the distal tip of the cannula for tissue manipulation.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a partial perspective view of an example embodiment of a needle for attachment to an ophthalmic surgical handpiece;

FIG. 2 is another partial perspective view of the needle of FIG. 1;

FIG. 3 is another partial perspective view of the needle of FIG. 1;

FIG. 4 is a partial cross-section view of another example embodiment of a needle for attachment to an ophthalmic surgical handpiece; and

FIG. 5 is a diagrammatic elevation view of an example embodiment of an ophthalmic surgical handpiece including the needle of FIG. 1.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

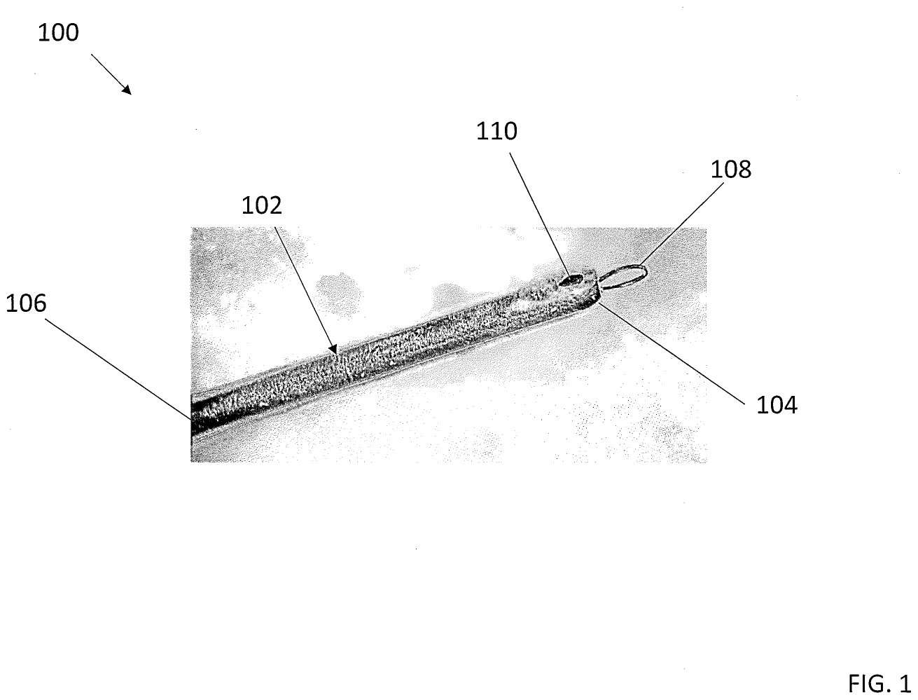

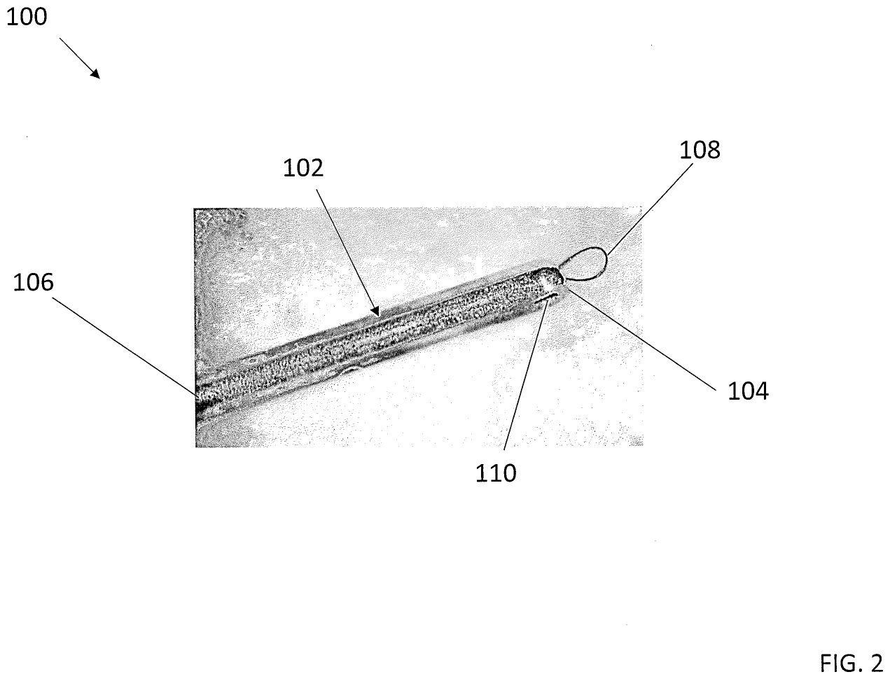

FIGS. 1-3 illustrate an example embodiment of a needle 100 (e.g., an ultrasonic needle for phacoemulsification or fragmentation) for attachment to an ophthalmic surgical handpiece (e.g., an ultrasonic handpiece, a fragmentation handpiece, a phacoemulsification handpiece, Bausch+Lomb's Vitesse™ Hypersonic Vitrectomy handpiece, etc.) including one or more aspects of the present disclosure. In the illustrated embodiment, the needle 100 includes a cannula 102 having a distal end 104 (e.g., a distal tip) and a proximal end 106. The needle 100 also includes a flexible member 108 (broadly, a tissue manipulation structure) coupled to the distal end 104 of the cannula 102 for tissue manipulation.

In the illustrated embodiment, the cannula 102 includes a port 110 that is in communication (e.g., in fluid communication) with a lumen (not shown in these views) extending through the cannula 102. Although only one port 110 is shown, in other embodiments, the cannula 102 may include additional ports within the scope of the present disclosure. As shown in FIG. 1, the port 110 is positioned adjacent to the distal end 104 of the cannula 102.

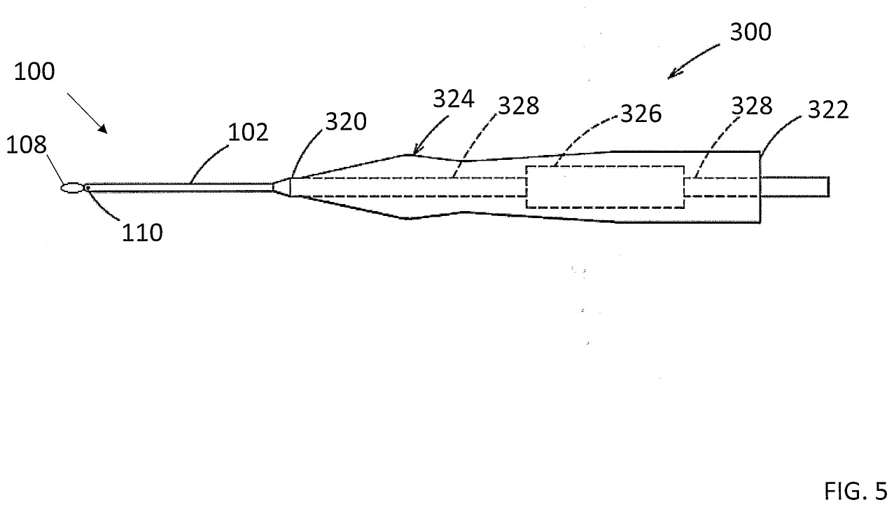

The proximal end 106 of the cannula 102 is configured (not shown) for attachment to an ophthalmic surgical handpiece (e.g., to a distal end of the handpiece) (see FIG. 5 for an example). For example, in some embodiments, the proximal end 106 includes a threaded connection for attachment to the handpiece. The handpiece generally includes a housing and a vibration source disposed within the housing for vibrating the distal tip 104 of the cannula 102 to assist in removal of tissue (e.g., vitreous, other tissues, etc.) from a patient's eye. The vibration source may cause the distal end 104 of the cannula 102 (broadly, the needle 100) to vibrate ultrasonically to emulsify affected tissue. An aspiration source may be connected to the housing to apply a negative pressure to the lumen and the aspiration port 110 to remove fluids and tissue from the eye. In this way, the ophthalmic surgical handpiece provides ultrasonic emulsification and aspiration of tissue from the eye through the cannula 102.

As described above, the flexible member 108 of the needle 100 is coupled to the distal end 104 of the cannula 102. As shown in FIGS. 1-3, the flexible member 108 is configured as a loop of flexible material. In the illustrated embodiment, the distal end 104 of the cannula 102 includes an opening into which the flexible member 108 is inserted (e.g., the ends of the loop are inserted into the opening, etc.). The flexible member 108 is secured within the opening at the distal end 104 and any gaps (e.g., within the opening between the flexible member 108 and the cannula 102, etc.) are filled to prevent leakage of fluids. For example, any gaps around the opening where the flexible member 108 is attached may be filled with a suitable material including a silicone seal or the like. In some embodiments, the opening at the distal end 104 of the cannula 102 is added to the cannula 102 through a secondary process (e.g., after forming the cannula 102). Alternatively, the cannula 102 is designed in some embodiments to be manufactured with the opening for the flexible member 108, where the opening is manufactured in the primary manufacturing process. The flexible member 108 is formed of a soft and/or flexible material, including polymers such as polyamide or silicone. Due to the material resiliency and small structure of the flexible member 108, the needle 100 is able to be inserted into an eye with minimal deformation of the flexible member 108 (e.g., such that the flexible member 108 generally retains its shape during insertion into the eye).

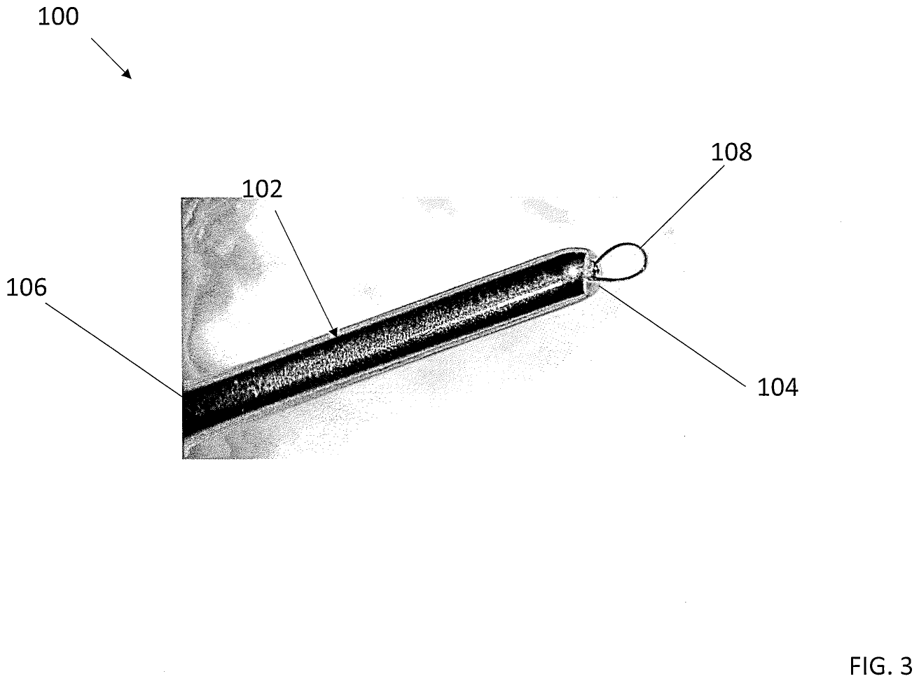

The flexible member 108, when configured as a loop, is generally formed in a round or oval shape. In the illustrated embodiment, the flexible member 108 is formed in a loop that is a teardrop shape, with the point of the teardrop coupled to the distal end 104 of the cannula 102. In particular, the flexible member 108 comprises a strand of material where each end of the strand is coupled to the distal end 104 of the cannula 102 (e.g., each end of the stand is coupled within the opening at the distal end 104 of the cannula 102 and sealed therein, as described above, etc.). In some embodiments, the flexible member 108 is pre-manufactured to a desired shape (e.g., a teardrop shape, etc.) and subsequently attached to the cannula 102, for example, where a closed loop is first formed and then attached to the cannula 102. While the flexible member 108 in this embodiment is depicted as a loop, it should be appreciated that in other embodiments, the flexible member 108 may comprise other shapes and/or configurations apart from a loop, including, for example, a button (see, e.g., FIG. 3) based on the desired tissue manipulation functionality of the flexible member 108. Moreover, the flexible member 108 is formed in a particular size and the size of the flexible member 108 is not adjustable (e.g., the circumference of the loop is not adjustable). In the illustrated embodiment, the flexible member 108 is positioned parallel to the cannula 102. In particular, the flexible member 108 extends from the distal end 104 of the cannula 102 in a direction parallel to a central longitudinal axis of the cannula 102. It should be appreciated that in other embodiments, the flexible member 108 may be positioned in other orientations, such as angled, perpendicular to the central longitudinal axis, etc. within the scope of the present disclosure.

The flexible member 108 is an additional structure positioned at the distal end 104 of the cannula 102 which serves as a cushion between the cannula 102 and retinal tissues within the eye during vibration of the needle 100. In this way, the flexible member 108 enhances the safety of the needle 100, for example, when the needle 100 is in close proximity to highly delicate parts of the eye, such as the retina and posterior capsule. In particular, because the flexible member 108 is formed of a soft and flexible material, the flexible member 108 is very gentle when in contact with the retina, with and without vibration of the cannula 102. Moreover, the flexible member 108 serves as a visual indicator of the distance of the distal end 104 of the cannula 102 from the targeted tissues. For example, the flexible member 108 is a visual indicator to a surgeon operating the needle 100 that the cannula 102 is approaching the retina. In particular, when the flexible member 108 contacts the retina, the flexible member 108 deforms in a manner that is visible or perceptible to the surgeon. A visual or perceptible deformation of the flexible member 108 against the retina acts as a warning that extra-precaution is warranted during the surgical maneuver. In some embodiments, the flexible member 108 is formed in a color that is readily perceptible during surgery, for example, blue or black (e.g., to contrast the retina and red blood cells that are commonly present in many disease states of the posterior cavity, etc.). The flexible member 108 also enables manipulation of tissue of a patient's eye during surgery, for example, as a lasso to grasp targeted and/or affected tissue. Additionally, when the cannula 102 is not energized (e.g., when the needle 100 is not being vibrated, etc.), the flexible member 108 enables retina membrane separation, pushing of detached retinal tissues to desired positions, and easier and safer removal of tissues via aspiration.

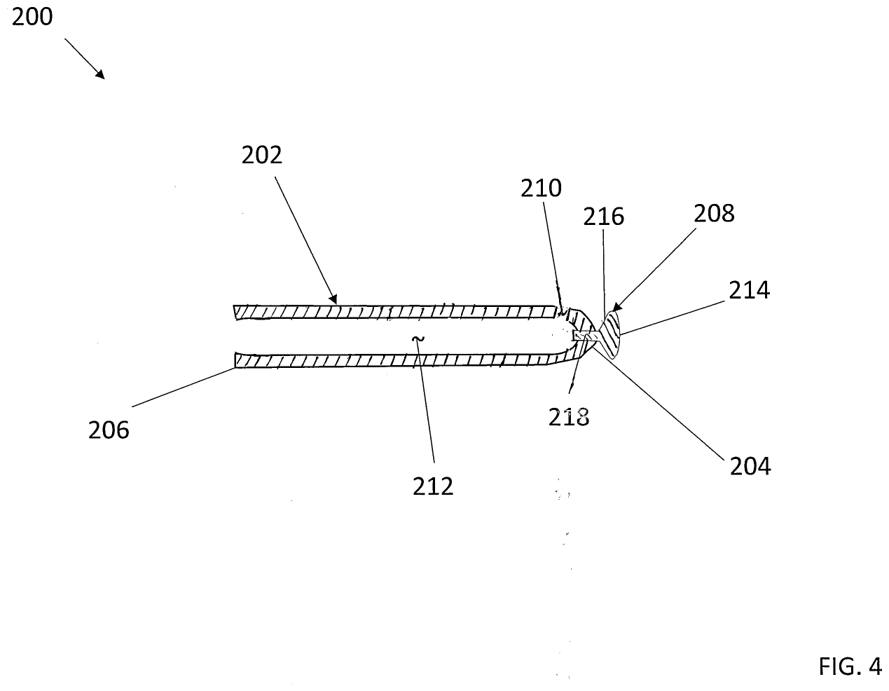

FIG. 4 illustrates another example embodiment of a needle 200 (e.g., an ultrasonic needle) for attachment to an ophthalmic surgical handpiece (e.g., an ultrasonic handpiece, a phacoemulsification handpiece, etc.) according to the present disclosure. The needle 200 of this embodiment is similar to the needle 100 previously described and illustrated in FIGS. 1-3. For example, the needle 200 of this embodiment again includes a cannula 202 having a distal end 204 (e.g., a distal tip) and a proximal end 206. The needle 200 also includes a flexible member 208 (broadly, a tissue manipulation structure) coupled to the distal end 204 of the cannula 202 for tissue manipulation.

In the illustrated embodiment, the cannula 202 includes a port 210 that is in communication (e.g., in fluid communication) with a lumen 212 extending through the cannula 202. Although only one port 210 is shown, in other embodiments, the cannula 202 may include additional ports within the scope of the present disclosure. As shown in FIG. 4, the port 210 is positioned adjacent to the distal end 204 of the cannula 202.

The proximal end 206 of the cannula 202 is configured for attachment to an ophthalmic surgical handpiece (e.g., to a distal end of the handpiece). For example, in some embodiments, the proximal end 206 includes a threaded connection (not shown) for attachment to the handpiece (not shown). The handpiece generally includes a housing and a vibration source disposed within the housing for vibrating the distal tip 204 of the cannula 202 to assist in removal of tissue (e.g., vitreous, other tissues, etc.) from a patient's eye. The vibration source may cause the distal end 204 of the cannula 202 (broadly, the needle 200) to vibrate ultrasonically to emulsify affected tissue. An aspiration source may be connected to the housing to apply a negative pressure to the lumen and the aspiration port 210 to remove fluids and tissue from the eye. In this way, the ophthalmic surgical handpiece provides ultrasonic emulsification and aspiration of tissue from the eye through the cannula 202.

As described above, the flexible member 208 of the needle 200 is coupled to the distal end 204 of the cannula 202. As shown in FIG. 4, the flexible member 208 is configured as a button of flexible material (e.g., a generally flat, circular projection from the distal end 204 of the cannula 202). In particular, the flexible member 208 includes a first surface 214 (e.g., a distal surface) which is positioned generally perpendicular to the cannula 202 (i.e., positioned perpendicular to a central longitudinal axis of the cannula 202). In the illustrated embodiment, the first surface 214 is curved, although it should be appreciated in other embodiments, the first surface 214 of the flexible member 208 may be flat, concave, faceted, etc. The flexible member 208 also includes a second surface 216 which, in the illustrated embodiment, is conical or tapered from the first surface 214 towards the distal end 204 of the cannula 202. In particular, the intersection of the first surface 214 and the second surface 216 of the flexible member 208 forms a lip (e.g., a rounded edge) which aids in tissue manipulation (e.g., lifting of membrane from the surface of the retina, etc.). It should be appreciated that in other embodiments, the second surface 216 may be flat, generally parallel to the first surface 214, etc. to form the lip without departing from the scope of the present disclosure.

The flexible member 208 also includes a base 218 which is inserted into an opening at the distal end 204 of the cannula 202. The base 218 of the flexible member 208 is secured within the opening at the distal end 204 and any gaps (e.g., within the opening between the flexible member 208 and the cannula 202, etc.) are filled to prevent leakage of fluids. For example, any gaps around the opening where the flexible member 208 is attached may be filled with a suitable material including a silicone seal or the like. The flexible member 208 is formed of a soft and/or flexible material, including polymers such as polyamide or silicone. Due to the material resiliency and small structure of the flexible member 208, the needle 200 is able to be inserted into an eye with no or minimal deformation of the flexible member 208 (e.g., such that the flexible member 208 generally retains its shape during insertion into the eye).

Similar to flexible member 108, the flexible member 208 is an additional structure positioned at the distal end 204 of the cannula 202 which serves as a cushion between the cannula 202 and retinal tissues within the eye during vibration of the needle 200. In this way, the flexible member 208 enhances the safety of the needle 200, for example, when the needle 200 is in close proximity to highly delicate parts of the eye, such as the retina and posterior capsule. In particular, because the flexible member 208 is formed of a soft and flexible material, the flexible member 208 is very gentle when in contact with the retina, with and without vibration of the cannula 202. Moreover, the flexible member 208 serves as a visual indicator of the distance of the distal end 204 of the cannula 202 from the targeted tissues. For example, the flexible member 208 is a visual indicator to a surgeon operating the needle 200 that the cannula 202 is approaching the retina. In particular, when the flexible member 208 contacts the retina, the flexible member 208 deforms in a manner that is visible or perceptible to the surgeon. A visual or perceptible deformation of the flexible member 208 against the retina acts as a warning that extra-precaution is warranted during the surgical maneuver. In some embodiments, the flexible member 208 is formed in a color that is readily perceptible during surgery, for example, blue or black (e.g., to contrast the retina and red blood cells that are commonly present in many disease states of the posterior cavity, etc.). The flexible member 208 also enables manipulation of tissue of a patient's eye, for example, for retina membrane separation, pushing of detached retinal tissues to desired positions, etc.

FIG. 5 illustrates an example embodiment of an ophthalmic surgical handpiece 300 (e.g., an ultrasonic handpiece, a fragmentation handpiece, a phacoemulsification handpiece, Bausch+Lomb's Vitesse™ Hypersonic Vitrectomy handpiece, etc.), including the needle 100 (with the flexible member 108) attached thereto, according to the present disclosure. While FIG. 5 illustrates the needle 100 being attached to the handpiece 300, other needles, such as needle 200, may be attached to the handpiece 300 in other embodiments. The handpiece 300 includes a distal end 320 and a proximal end 322. As shown, the needle 100 is attached to the distal end 320 of the handpiece 300. For example, in some embodiments, the distal end 320 of the handpiece 300 may include a threaded connection for attachment to the needle 100. The handpiece generally includes a housing 324 and a vibration source 326 disposed within the housing 324 for vibrating the needle 100 (e.g., the distal tip of the cannula 102, etc.) to assist in removal of tissue (e.g., vitreous, other tissues, etc.) from a patient's eye. The vibration source 326 causes the needle 100 to vibrate ultrasonically, for example, to emulsify affected tissue. The handpiece 300 includes an aspiration path 328 and an aspiration source may be connected to the aspiration path 328 to apply a negative pressure to the aspiration port 110 of the needle 100 to remove fluids and tissue from the eye through the aspiration path 328. In this way, the handpiece 300, in combination with the needle 100, provides ultrasonic emulsification and aspiration of tissue from the eye.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” as well as the phrase “at least one of” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper”, “lower” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the present disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the present disclosure, and all such modifications are intended to be included within the scope of the present disclosure.

What is claimed is:

1. An ultrasonic needle for attachment to an ophthalmic surgical handpiece, the ultrasonic needle comprising:
a cannula having a proximal end and a distal end, the proximal end configured for attachment to an ophthalmic surgical handpiece, the distal end including a tip with at least one port in communication with a lumen extending through the cannula; and
a flexible member coupled to the distal end of the cannula for tissue manipulation.

2. The ultrasonic needle of claim 1, wherein the flexible member is a loop of flexible material.

3. The ultrasonic needle of claim 2, wherein the loop comprises a strand having a first end and a second end, wherein the first end and the second end are coupled to the distal end of the cannula.

4. The ultrasonic needle of claim 2, wherein the loop is parallel to a central longitudinal axis of the cannula.

5. The ultrasonic needle of claim 2, wherein the loop is teardrop shaped.

6. The ultrasonic needle of claim 1, wherein the flexible member is a button of flexible material.

7. The ultrasonic needle of claim 6, wherein the button includes a distal surface, a tapered surface, and a lip between the distal surface and the tapered surface.

8. The ultrasonic needle of claim 7, wherein the distal surface is perpendicular to a central longitudinal axis of the cannula.

9. The ultrasonic needle of claim 1, wherein the flexible member is polyamide.

10. The ultrasonic needle of claim 1, wherein the flexible member is silicone.

11. An ophthalmic surgical device comprising:
a housing having a distal end;
a cannula attached to the distal end of the surgical device and having a distal tip with at least one aspiration port;
a vibration source disposed within the housing for vibrating the distal tip of the cannula; and
a flexible member coupled to the distal tip of the cannula for tissue manipulation.

12. The ultrasonic needle of claim 11, wherein the flexible member is a loop of flexible material.

13. The ultrasonic needle of claim 12, wherein the loop comprises a strand having a first end and a second end, wherein the first end and the second end are coupled to the distal end of the cannula.

14. The ultrasonic needle of claim 12, wherein the loop is parallel to a central longitudinal axis of the cannula.

15. The ultrasonic needle of claim 12, wherein the loop is teardrop shaped.

16. The ultrasonic needle of claim 11, wherein the flexible member is a button of flexible material.

17. The ultrasonic needle of claim 16, wherein the button includes a distal surface, a tapered surface, and a lip between the distal surface and the tapered surface.

18. The ultrasonic needle of claim 17, wherein the distal surface is perpendicular to a central longitudinal axis of the cannula.

19. The ultrasonic needle of claim 11, wherein the flexible member is polyamide and/or silicone.

20. The ophthalmic surgical device of claim 11, wherein the housing and the vibration source are a part of a phacoemulsification surgical handpiece.
